(19) United States
(12) Patent Application Publication
(10) Pub. No.: US 2011/0044914 A1
(43) Pub. Date: Feb. 24, 2011

(54) CLEANING COMPOSITIONS AND METHODS

(75) Inventors: Rajnish Kohli, Hillsborough, NJ (US); Richard Scott Robinson, Belle Mead, NJ (US); Ralph Peter Santarpia, III, Edison, NJ (US); James R. Brown, Edison, NJ (US)

Correspondence Address: COLGATE-PALMOLIVE COMPANY, 909 RIVER ROAD, PISCATAWAY, NJ, 08855, US

(73) Assignee: Colgate -Palmolive Company, New York, NY (US)

(21) Appl. No.: 12/866,759

(22) PCT Filed: Feb. 6, 2009
(86) PCT No.: PCT/US09/33310

§ 371 Date: Aug. 9, 2010

Related U.S. Application Data
(60) Provisional application No. 61/027,423, filed on Feb. 8, 2008.

(51) Int. Cl. A61K 31/198; A01N 37/44; A61K 31/4172; A01N 43/50; A01N 47/28; A01N 47/44; A61K 33/00; A01N 59/00; A61K 33/14; A61K 8/44; A61K 8/49; A61Q 11/00; A61P 1/02; A01P 1/00

(52) U.S. Cl. 424/44

(57) ABSTRACT

The present invention is directed to effervescent cleaning compositions comprising a basic amino acid.

This application claims the benefit of U.S. patent application Ser. No. 61/027,423 filed Feb. 8, 2008, the contents of which are incorporated herein by reference.

### BACKGROUND OF THE INVENTION

Effervescent cleaners for dental devices are well known in the art, and generally come in the form of a powder or tablet which “fizzes” when added to water. Such effervescent cleaners have been utilized in various arts for multiple purposes. For example, POLIDENT by, GlaxoSmithKline, and EFFERDENT from Pfizer is used to clean dental devices. Dental devices are instruments intended for repeated use in the oral cavity by repeated removal and insertion, and are well known in the art. Dental devices include tooth brushes, tongue scrapers, dental floss, dental picks, mouth guards, dentures, and orthodontic appliances, such as dentures, e.g., false teeth, dental plates and bridges, and orthodontic corrective devices, e.g., retainers. A common problem with dental devices is that they become stained or absorb odors from the oral cavity. Such odors may be caused by oral microbial flora, and thus the cleaning of dental devices is required, almost daily. Existing effervescent cleaners, however, are principally directed to treating the dental device, rather than the oral health of the user.

### SUMMARY OF THE INVENTION

The present invention is directed to compositions and methods to clean dental devices and improve the oral health of the user.

Arginine and other basic amino acids have been proposed for use in oral care and are believed to have significant benefits in combating cavity formation and tooth sensitivity. It is believed that basic amino acids in the oral cavity are metabolized by certain types of bacteria, e.g., S. sanguis which are not cariogenic and which compete with cariogenic bacteria such as S. mutans, for position on the teeth and in the oral cavity. The arginolytic bacteria can use arginine and other basic amino acids to produce ammonia, thereby raising the pH of their environment, while cariogenic bacteria metabolize sugar to produce lactic acid, which tends to lower the plaque pH and demineralize the teeth, ultimately leading to cavities. Arginine and other basic amino acids therefore protect the teeth from cariogenic bacteria, and moreover play an important role in promoting remineralization of the teeth by calcium and phosphate.

Effervescence is usually produced by the reaction of an acid with a carbonate salt, to release carbon dioxide. For example, citric acid may react with sodium bicarbonate to form carbon dioxide, water and sodium citrate.

By “soluble carbonate salt” is meant any salt formed by carbonic acid or dissolved carbon dioxide which is sufficiently soluble to react with the acid in the concentrations provided. In aqueous solution, the carbonate ion, bicarbonate ion, carbon dioxide, and carbonic acid form a dynamic equilibrium. The term “carbonate” as used herein thus encompasses bicarbonate (HCO₃) and carbonate (CO₃²⁻) forms and mixtures thereof. Soluble carbonate salts thus include, e.g., potassium carbonate, potassium bicarbonate, sodium carbonate, and sodium bicarbonate. The invention thus includes Composition 1.0, an effervescent dissolvable solid (e.g. power, granulate or tablet) comprising
a. an acid source and a soluble carbonate salt; and
b. a basic amino acid in free or salt form.

In one embodiment, the basic amino acid (b) is in bicarbonate salt form and so can form all or part of the carbonate salt of (a). The invention further includes the following Compositions:
1.1 Composition 1.0 wherein the basic amino acid is arginine, lysine, citrullene, ornithine, creatine, histidine, diaminobutanoic acid, diaminoproprionic acid, salts thereof and/or combinations thereof.
1.2 Composition 1.0 or 1.1 wherein the basic amino acid has the L-configuration.
1.3 Any of the preceding compositions is provided in the form of a salt of a di- or tri-peptide comprising the basic amino acid.
1.4 Any of the preceding compositions wherein the basic amino acid is arginine.
1.5 Any of the preceding compositions wherein the basic amino acid is L-arginine.
1.6 Any of the preceding compositions wherein the salt of the basic amino acid is a carbonate.
1.7 Any of the preceding compositions wherein the salt of the basic amino acid is a bicarbonate.
1.8 Any of the preceding compositions in the basic amino acid salt is arginine bicarbonate.
1.9 Any of the preceding compositions wherein the basic amino acid is present in an amount corresponding to about 0.1 wt. % to about 50 wt. % of the total composition weight, the weight of the basic amino acid being calculated as free base form.
1.10 Any of the preceding compositions wherein the acid source is selected from citric acid, malic acid, tartaric acid, adipic acid, succinic and fumaric acid, and combinations thereof.
1.11 Any of the preceding compositions wherein the carbonate salt is selected from sodium bicarbonate, potassium bicarbonate, sodium carbonate, arginine bicarbonate, and potassium carbonate.
1.12 Any of the preceding compositions which produces carbon dioxide when dissolved in a solvent, e.g., water.
1.13 Any of the preceding compositions comprising a bleaching agent.
1.14 Any of the preceding compositions comprising an alkali metal percarbonate, perborate, persulfate, perpyrophosphate and monopersulfate.
1.15 Any of the preceding compositions further comprising fluoride, or a fluoride ion source.
1.16 Any of the preceding compositions further comprising an antiseptic or antimicrobial.

In one embodiment of the present invention, Composition 2.0 is provided comprising any of compositions 1.0-1.16 in the form of a tablet.

The present invention also includes the following compositions:
2.1 Of composition 2.0 further comprising a lubricant
2.2 Of composition 2.0 or 2.1 comprising a lubricant selected from magnesium stearate, sodium benzoate, polyethylene glycol, adipic acid, and combinations thereof.
2.3 Of composition 2.0-2.2 further comprising a binder.
2.4 Of composition 2.0-2.3 comprising a binder selected from dextrose, sorbitol, xyitol, lactose, and combinations thereof

The present invention also encompasses method 3.0, a method to clean a dental device, using a composition as described above,
a. to deliver arginine to the mouth
b. to reduce the biofilm and bacterial attachment on the device
c and/or to reduce the population of cariogenic bacteria on the device comprising adding a Composition of the Invention an aqueous solvent, immersing a dental instrument into the resulting solution for an effective time to clean said dental instrument, and applying or using said dental instrument in the oral cavity.

The method may further be used to promote oral health in the user of the device, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5, (xi) reduce plaque accumulation, (xii) clean the teeth and oral cavity, (xiii) immunize the teeth against cariogenic bacteria, (xiv) reduce erosion, (xv) enhance systemic health, and/or (xvi) treat or inhibit dry mouth.

Other embodiments of the present invention will be apparent to one of skill in the art.

### DETAILED DESCRIPTION OF THE INVENTION

Effervescence generally results from the reaction of a soluble acid and soluble base in water to produce carbon dioxide or oxygen. Such acids and bases are well known in the art. Such acids include soluble organic acids, such as citric, malic, tartaric, adipic, succinic and fumaric acid. Such acids are generally in salt form prior to addition to water. Bases include basic salts of amino acids, and water soluble carbonates and bicarbonates, such as sodium bicarbonate, potassium bicarbonate, sodium carbonate, and potassium bicarbonate. Such bases are generally in salt form prior to addition to water. It has been surprisingly found that basic amino acids salts may not only be used as a basic salt, but such basic amino acid salts also impart benefits to the oral cavity.

The cleaning compositions of the present invention comprise cleaning and oxidizing agents, such as chlorine generating compounds such as dichloroisocyanurates, as well as chlorine free bleaches, including alkali metal percarbonate, perborate, persulfate, perpyrophosphate and monopersulfate. Anhydrous perborates, such as sodium perborate be utilized to cause effervescence by releasing oxygen, which in addition to causing effervescence contributes, also aids in the bleaching of the dental instrument. Perborates are known in the art, and include calcium perborate, ammonium perborate, magnesium perborate, and anhydrous potassium perborate. Perborates may comprise from about 5% to about 25% total weight of the composition. Monoperphthalic acid may also be useful as a bleaching agent of the denture cleansing composition, and a perborate or monopersulfate, such as potassium monopersulfate, may be is used in conjunction with a monoperphthalic acid. One preferred bleaching agent is potassium peroxymonosulphate, which may form from about 2% to about 15% weight of the composition. Other cleaning agents include alkaline hypochlorites and alkaline peroxides, and β-1,3-glucanase.

Without intending to be bound by a particular theory, it is believed that basic amino acids in the oral cavity are metabolized by certain types of bacteria, e.g., S. sanguis which are not cariogenic and which compete with cariogenic bacteria such as S. mutans, for position on the teeth and in the oral cavity. The arginolytic bacteria can use arginine and other basic amino acids to produce ammonia, thereby raising the pH of their environment, while cariogenic bacteria metabolize sugar to produce lactic acid, which tends to lower the plaque pH and demineralize the teeth, ultimately leading to cavities. It is believed that use of a Composition of the Invention may lead to a relative increase in the arginolytic bacteria and a relative decrease in the cariogenic bacteria, resulting in a higher plaque pH.

The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of about 7 or greater. Accordingly, basic amino acids include, but are not limited to, arginine, lysine, citrullene, ornithine, creatine, histidine, diaminobutanoic acid, diaminoproprionic acid, salts thereof or combinations thereof. In a particular embodiment, the basic amino acids are selected from arginine, citrullene, and ornithine, preferably, arginine, for example, 1-arginine.

The compositions of the invention are used in the mouth, and optionally may be ingested, and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion. A preferred salt is a bicarbonate, e.g., arginine bicarbonate.

In various embodiments, the basic amino acid is present in an amount of about 0.1 wt. % to about 50 wt. % of the total composition weight, about 1 wt. to about 40 wt. % of the total composition weight, for example about 1.5 wt. %, 10 wt. %, 20 wt. %, or 30 wt. % of the total composition weight.

If the compositions of the present invention may optionally include fluoride, or a fluoride ion source e.g., when formulated to be dissolved in a solvent to be used as a mouthwash. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., incorporated herein by reference. Representative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. Thus, such effervescence powders may also contain a source of fluoride ions or fluorine-providing ingredient in amounts sufficient to supply about 25 ppm to about 25,000 ppm of fluoride ions, generally at least about 500 ppm, e.g., about 500 to about 2000 ppm, e.g., about 1000 to about 1600 ppm, about 1450 ppm. Fluoride ion sources may be added to the compositions of the invention at a level of about 0.01 wt. % to about 10 wt. % in one embodiment or about 0.03 wt. % to about 5 wt. %, and in another embodiment about 0.1 wt. % to about 1 wt. % by weight of the composition in another embodiment. Weights of fluoride salts to provide the appropriate level of fluoride ion will obviously vary based on the weight of the counter ion in the salt.

The compositions of the present invention also comprise antiseptics antimicrobial compounds, e.g.; triclosan, herbal extracts and essential oils (e.g. rosemary extract, thymol, menthol, eucalyptol, methyl salicylate), bisguanide antiseptics (e.g., chlorhexidine, alexidine or octenidine), quaternary ammonium compounds (e.g., cetylpyridinium chloride), phenolic antiseptics, hexetidine, povidone iodine, delmopinol, salifluor, metal ions (e.g., zinc salts, for example, zinc citrate), sanguinarine, propolis, and antibiotics. Such antiseptics and antimicrobial compounds are desirable when the effervescent powers of the present invention are formulated to dissolve in a solvent to form a mouthwash.

The compositions of the present invention may also include one or more flavoring agents. Flavoring agents which are used in the practice of the present invention include, but are not limited to, essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint. Various acids and bases to produce effervescence may also be flavoring agents, such as citric acid and malic acid.

The flavoring agent is incorporated in the oral composition at a concentration of about 0.1 to about 5% by weight and about 0.5 to about 1,5% by weight. The dosage of flavoring agent in the individual oral care composition dosage (i.e., a single dose) is about 0.001 to 0.05% by weight and in another embodiment about 0.005 to 0.015% by weight

The cleaning compositions of the present invention may be compressed into a tablet form, e.g., to create a single dose format to be added to a solvent. Methods of producing tablets, and general tablet compositions are well known in the art. Tablets generally contain a binder, which are known by those of skill in the art. Preferably, the binders are soluble, and include, e.g., dextrose, sorbitol, xylitol, and lactose. Preferably, the amount of binder allows for the tablet to be hard enough to handle, soft enough to disintegrate when introduced into a solvent, and dry enough to be stable.

Tablets may also comprise a lubricant to aid in expulsion of the table from a press. Such lubricants arc known by those of skill in the art, and include magnesium stearate, sodium benzoate, polyethylene glycol, and adipic acid.

1. An effervescent dissolvable solid composition comprising:
an acid source; and
a soluble carbonate salt of a basic amino acid.

2. The composition of claim 1 wherein the basic amino acid is selected from arginine, lysine, citrullene, ornithine, creatine, histidine, diaminobutanoic acid, diaminoproprionic acid, salts thereof and combinations thereof.

3. The composition of claim 1 wherein the basic amino acid has the L-configuration.

4. The composition of claim 1 wherein the basic amino acid is arginine.

5. (canceled)

6. The composition of claim 1 wherein the soluble carbonate salt of the basic amino acid is arginine bicarbonate.

7. (canceled)

8. The composition of claim 1 wherein the basic amino acid is present in the amount of about 0.1 wt. % to about 50 wt. % of the total composition weight, the weight of the basic amino acid being calculated as free base form.

9. The composition of claim 1 wherein the acid source is selected from citric acid, malic acid, tartaric acid, adipic acid, succinic acid and fumaric acid.

10. The composition of claim 1 further comprising an additional soluble carbonate salt selected from sodium bicarbonate, potassium bicarbonate, arginine bicarbonate, sodium carbonate, and potassium carbonate.

11. The composition of claim 1 which produces carbon dioxide when dissolved in a solvent.

12. The composition of claim 1, further comprising a bleaching agent.

13. The composition of claim 1, further comprising an alkali metal percarbonate, perborate, persulfate, perpyrophosphate and monopersulfate.

14. The composition of claim 1, further comprising a fluoride ion source.

15. The composition of claim 1, further comprising an antiseptic or antimicrobial.

16. The composition of claim 1, wherein the composition is in a powder or granulate form.

17. The composition of claim 1, wherein the composition is in the form of a tablet.

18. (canceled)

19. The composition of claim 1 further comprising a surfactant.

20. The composition of claim 19 when the surfactant is selected from sodium lauryl sulfate and sodium ether lauryl sulfate.

21. A method for inhibiting or reducing the growth of cariogenic bacteria on a dental instrument comprising
adding the composition of claim 1 to an aqueous solvent, and
immersing a dental instrument into the resulting solution for an effective time to clean said dental instrument.

22. (canceled)

23. A method to
(i) reduce or inhibit formation of dental caries,
(ii) reduce, repair or inhibit pre-carious lesions of the enamel, e.g., as detected by quantitative light-induced fluorescence (QLF) or electrical caries measurement (ECM),
(iii) reduce or inhibit demineralization and promote remineralization of the teeth,
(iv) reduce hypersensitivity of the teeth,
(v) reduce or inhibit gingivitis,
(vi) promote healing of sores or cuts in the mouth,
(vii) reduce levels of acid producing bacteria,
(viii) increase relative levels of arginolytic bacteria,
(ix) inhibit microbial biofilm formation in the oral cavity,
(x) raise and/or maintain plaque pH at levels of at least pH 5.5,
(xi) reduce plaque accumulation,
(xii) clean the teeth and oral cavity,
(xiii) immunize the teeth against cariogenic bacteria,
(xiv) reduce erosion,
(xv) promote systemic health, and/or
(xvi) treat or inhibit dry mouth;
comprising administering the composition of claim 1 to the oral cavity of a subject in need thereof.

24. The method of claim 21, further comprising the step of rinsing the dental instrument in water.

25. The method of claim 21 wherein the dental instrument is selected from the group consisting of tooth brushes, tongue scrapers, dental floss, dental picks, mouth guards, dentures, orthodontic appliances, orthodontic corrective devices.

26. The composition of claim 14, wherein said fluoride ion source is selected from stannous fluoride, sodium fluoride, potassium fluoride, sodium monofluorophosphate, sodium fluorosilicate, ammonium fluorosilicate, amine fluoride, ammonium fluoride, and combinations thereof.

27. The composition of claim 1, further comprising a potassium ion source.

28. The composition of claim 27, wherein said potassium ion source is selected from potassium nitrate and potassium chloride.
